(19) United States
(12) Patent Application Publication
(10) Pub. No.: US 2017/0145372 A1
(43) Pub. Date: May 25, 2017

(54) Embryo Incubator Incorporating Temperature Control

(71) Applicant: Kivex Biotec A/S, Birkerød (DK)

(72) Inventors: Niels Stengaard Hansen, Birkerød (DK); Ronny Janssens, Brussel (BE)

(73) Assignee: Kivex Biotec A/S, Birkerod (DK)

(21) Appl. No.: 15/321,303

(22) PCT Filed: Jun. 29, 2015
(86) PCT No.: PCT/DK2015/050190

§ 371 Date: Dec. 22, 2016

(30) Foreign Application Priority Data
Jun. 27, 2014 (DK) PA 2014 70396

(51) Int. Cl. C12M 1/00; C12M 1/12; C12M 1/34; C12M 1/36; C12M 3/00; C12M 1/02

(52) U.S. Cl. CPC C12M 41/14; C12M 21/06; C12M 41/22; C12M 41/34; C12M 41/40; C12M 41/48; C12M 37/00; C12M 41/26

(57) ABSTRACT

The invention relates to an incubator having a chamber for cultivating embryos and a gas control system and a temperature control system for providing respectively a predefined atmosphere and a predefined temperature in the incubating chamber. One embodiment relates to an incubator for incubating embryos, comprising one or more incubating chambers adapted to contain the embryo(s), a gas distribution system in fluid communication with the incubating chamber(s), configured to sustain a predefined level of O₂ and/or CO₂ in said incubating chamber(s), and a temperature control system, configured to provide said incubating chamber(s) with a predefined temperature.

The invention relates to an incubator having a chamber for cultivating embryos and a gas control system and a temperature control system for providing respectively a predefined atmosphere and a predefined temperature in the incubating chamber.

### BACKGROUND OF INVENTION

Infertility affects more than 80 million people worldwide. It is estimated that 10% of all couples experience primary or secondary infertility. In vitro fertilization (IVF) is an elective medical treatment that may provide a couple who has been otherwise unable to conceive a chance to establish a pregnancy. It is a process in which eggs (oocytes) are taken from a woman's ovaries and then fertilized with sperm in the laboratory. The embryos created in this process are then placed into the uterus for potential implantation. In between fertilization (insemination) and transfer, the embryos are typically stored in incubators for 2-6 days wherein the developmental conditions are optimized by emulating the conditions in the uterus. Thus, when incubating embryos it is of utmost importance to be able to maintain a constant and predefined atmosphere in the actual incubation chamber, and to control the temperature in the incubation chamber.

### SUMMARY OF INVENTION

A basic requirement to an incubator for incubating embryos is that the embryos can be easily placed and removed from the incubating chamber, and that the environmental conditions within the incubating chamber can be sustained, controlled, and quickly recovered upon interruptions. The present disclosure relates to an incubator for incubating embryos, comprising one or more incubating chambers adapted to contain the embryo(s), a gas distribution system in fluid communication with the incubating chamber(s), configured to sustain a predefined level of O₂ and/or CO₂ in said incubating chamber(s), and a temperature control system, configured to provide said incubating chamber(s) with a predefined temperature.

### DESCRIPTION OF DRAWINGS

FIG. 1 shows a programmed temperature profile for a daily temperature cycle, such as the basal body temperature.

FIG. 2 is a principal diagram of one embodiment of the gas distribution system of the presently disclosed incubator.

FIG. 3 shows an exemplary incubator with 10 incubation chambers, a preparation chamber, and a touch screen as part of a user interface, as seen from the outside.

FIG. 4 shows a screen shot from a user interface of one embodiment of the presently disclosed incubator.

FIGS. 5a-b show an exemplary incubator with a top unit and bottom unit in open (FIG. 5a) and closed (FIG. 5b) configuration.

FIG. 6 is a top view of an exemplary incubator bottom unit.

FIGS. 7a-c show an exemplary heating element where no or reduced electromagnetic radiation is emitted to the surroundings, consisting of a foil-shaped heating element, wherein the wires are configured so that the electromagnetic induction is eliminated.

FIG. 7a is a principle sketch illustrating the reduction in electromagnetic radiation, when wires with opposite directioned currents are positioned in vicinity or adjacent of each other.

FIG. 7b shows an exemplary foil heating element, where wires with opposite current directions are positioned superjacent to each other on either sides of an insulating foil, and the wire on the top side of the foil is placed in a meandering pattern that is symmetrical to the wire on the bottom side of the foil. The sketch in the top of FIG. 7b shows a cross-section of the heating element, the middle sketch shows the pattern on the top surface, and the bottom sketch shows the symmetrical pattern on the bottom side.

FIG. 7c shows an exemplary kit of components for a heating element, and a cross-section of the assembled kit is shown in the corner of FIG. 7c. The heating element comprises a wire of brass that is positioned on a PET foil. The wire is forming a continuous circuit that is geometrically positioned in two circuit halves that are mirror images such that by folding the PET foil, the two circuit halves will be superjacent in a symmetrical pattern with superjacent wire sections having opposite current flow. A PET layer is sandwiched in-between the two folded halves and mounted with an adhesive, and an additional PET layer with adhesive may is adhered to the bottom foil.

### DETAILED DESCRIPTION OF THE INVENTION

To ensure optimized developmental conditions for embryos cultivated in incubators, the environmental conditions in the incubation chamber must be regulated, sustained and controlled. The environmental conditions include the oxygen concentration (or oxygen tension) in the incubation chamber, pH in the medium containing the embryos, carbon dioxide concentration, and the temperature in the incubation chamber.

In the present disclosure the term incubating chamber is used synonymously with incubation chamber and culture chamber.

The present disclosure relates to an incubator for incubating embryos, comprising:

one or more incubating chambers adapted to contain the embryo(s),

a gas distribution system in fluid communication with the incubating chamber(s), configured to sustain a predefined level of O₂ and/or CO₂ in said incubating chamber(s), and

a temperature control system, configured to provide said incubating chamber(s) with a predefined temperature.

In one embodiment of the invention, the incubator comprises a plurality of incubating chambers, such as 10 culture chambers, similar to a previous incubator of the type G185 from the applicant.

In a further embodiment of the invention, the incubating chamber(s) is suitable for positioning of holding means for at least one embryo, e.g. microscopy slides, or similar inserts or culture dishes, such as disposable dishes containing growth media.

Temperature control systems typically comprise at least one heating element, at least one temperature sensor, and at least one programmable temperature regulator. The ability of the system to provide a predefined temperature and a uniform temperature distribution within a volume will depend on the heating elements and their geometrical arrangement. To minimize and avoid temperature gradients within the incubating chamber, one embodiment of the invention comprises that each incubating chamber is surrounded by at least one heating element, e.g. the heating element(s) may be implemented or incorporated in the incubator such that each incubation chamber is surrounded by at least one heating element. In a further embodiment, each incubating chamber has a box shape with at least one heating element implemented within all six sides, wherein one side may optionally be a closure, such as a lid.

A common heating element converts electricity into heat through resistive heating. The electric current will generate an electromagnetic field, and it is known that electromagnetic radiation may affect the cultivation of embryos. Thus, in one embodiment of the invention, the incubator comprises incubating chambers comprising at least one heating element configured such that electromagnetic radiation from the heating element is reduced or eliminated.

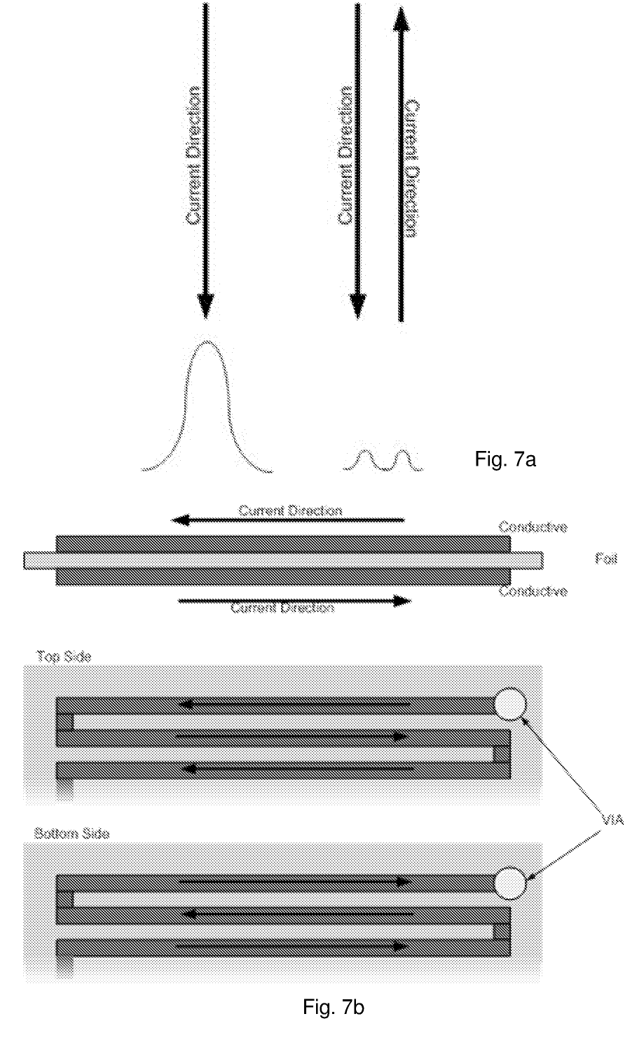

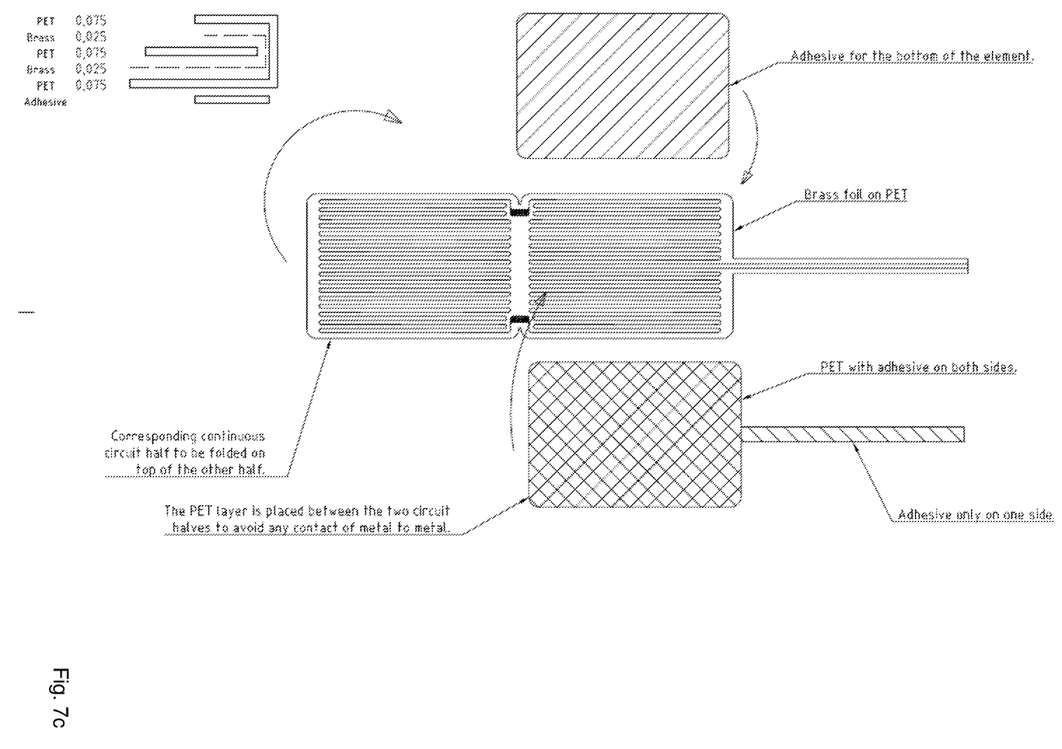

One way to reduce the electromagnetic radiation is to apply the principle of the resultant electromagnetic field of adjacent wires carrying current in opposite direction, as illustrated in FIG. 7a. In a further embodiment of the invention, at least one of the heating elements therefore comprise at least one essentially planar foil of insulating material on which an electrically conducting wire is positioned on, or contained in, a pattern that is configured to reduce or eliminate the electromagnetic induction or radiation to the surroundings.

In a further embodiment, the at least one heating element comprises a foil of insulating material that is sandwiched between at least two foil parts containing an electrically conducting wire, and wherein the wire sections of the at least two foil parts are configured to be essentially superjacent. In a further embodiment, current is applied to the heating element such that the direction of the current is opposite in the superjacent wire sections across the sandwiched insulating layer, thereby minimizing the resultant electromagnetic field, as illustrated in FIG. 7b. In a further embodiment of the invention, the electrically conducting wire is positioned in a meandering pattern as exemplified in FIG. 7b.

In a further embodiment of the invention, the at least one heating element is assembled by folding a foil containing a continuous electrically conducting wire around an insulating layer such that the insulating layer is sandwiched between the parts of the foil containing the wire. In a further embodiment, the heating element is assembled from a foil to be folded, which contains a wire that is positioned in a pattern containing two circuit halves that are mirror images, such that when the foil is folded around the mirror line, the two circuit halves become superjacent, and the direction of the current will be opposite in the superjacent sections. In a further embodiment, the folded foil and the sandwiched foil are assembled and fixed using a mean for adhesion. In a further embodiment of the invention, the heating element is assembled from the folded foil and sandwiched layer, and at least one additional layer of insulating material, wherein means for adhesion are applied on at least one of the surfaces of said additional layer. An example of the kit of parts for assembling the heating element, and a cross-section of the assembled parts, is illustrated in FIG. 7c.

The embodiments of the heating element may comprise a wire that may be of any conducting material, such as brass, and the insulating foil may be of any insulating material that can be shaped as a foil, such as PET, and the foils may be assembled using any suitable means for adhesion.

For embryos stored in an incubator, the cultivation or development conditions are optimized by emulating the conditions in the human body and the uterus. A controlled temperature in the incubation chamber is therefore critical, and may be obtained by the temperature control system. The temperature of the human body is known to be non-constant, i.e. to have a profile. Cyclic temperature variation of the human body is a known phenomenon. There is for example a diurnal variation, wherein the lowest body temperature (the basal body temperature) is attained during sleep at night. This cycle will in this document be referred to as the basal body temperature cycle. For women, a cyclic temperature variation over the menstrual cycle related to ovulation, is also occurring.

By incorporating an improved and programmable temperature control system, an incubator which simulates the conditions in the uterus more precisely compared to the presently available incubators is obtained. In one embodiment of the invention, the temperature control system is configured to provide the incubating chamber(s) with a predefined temperature profile, which optionally can be a profile with cyclic temperature variation, and where the cycle optionally can be repeated. In a further embodiment, the cyclic temperature profile can be set to simulate biological temperature variations, such as the daily variation known as the basal body temperature cycle.

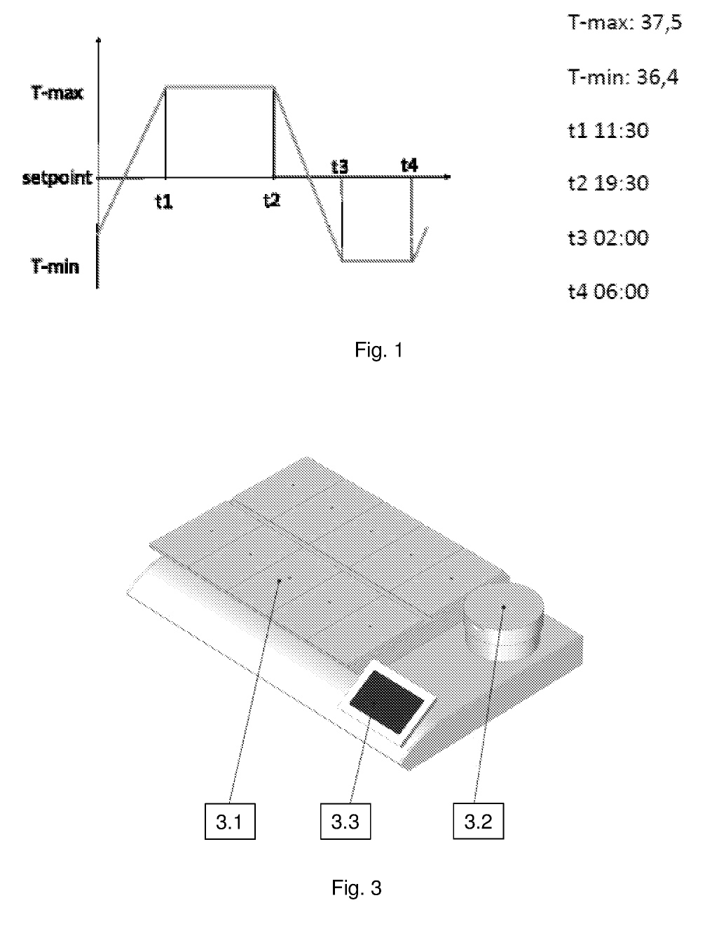

In a further embodiment, the temperature control system is configured to provide the incubating chamber(s) with a cyclic variation in temperature, controlled by a predefined maximum temperature (Tmax) and minimum temperature (Tmin), and a temperature ramp by piecewise linear changes defined by Tmax, Tmin, a start time and an end time for ramping, and with discrete incremental steps of 0.1 degree celcius. In a further embodiment a temperature cycle is defined by 24 hours duration, Tmax, Tmin, and four predefined temperature change times, and wherein said cycle is repeated automatically. In a further embodiment of the invention, a 24 hours cycle is defined by a Tmax of 37.5 C, Tmin of 36.4 C, and Tmax in the period from 11.30 AM to 7.30 PM, and Tmin in the period from 2.00 AM to 6.00 AM. An example of such a 24 hours cycle is illustrated in FIG. 1, where t1, t2, t3 and t4 indicate the predefined times for temperature changes.

The capacity of an incubator can be increased and the risk of cross contamination or mix up from patient to patient can be minimized, by incorporating separate incubating chambers. In one embodiment of the invention, the incubator comprises a plurality of incubating chambers, and the incubator may be configured such that the temperature in each chamber can be controlled independently. In a further embodiment of the invention, the incubator is configured such that a cyclic temperature variation in each chamber can be controlled independently. In a further embodiment, the incubator is configured such that a cyclic temperature variation will be the same in all of the incubating chambers, i.e. controlled dependently.

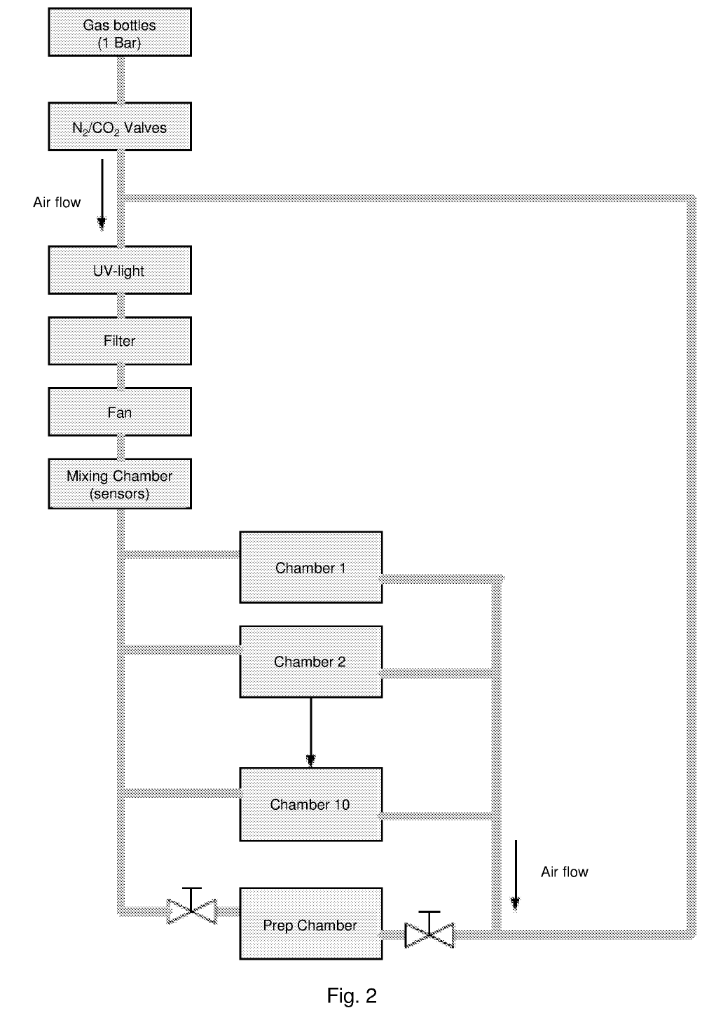

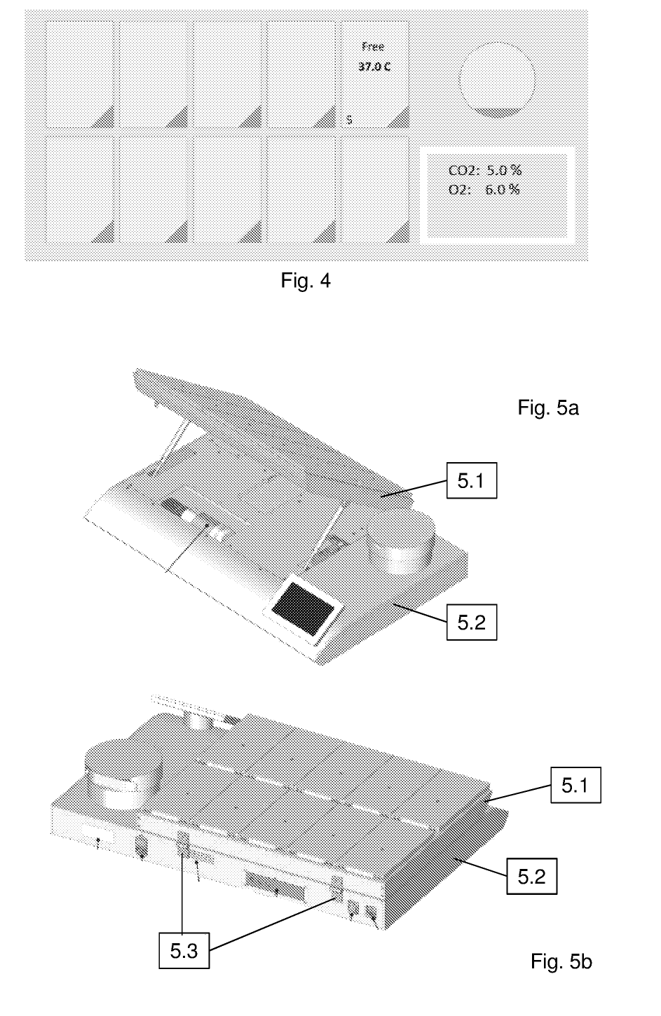

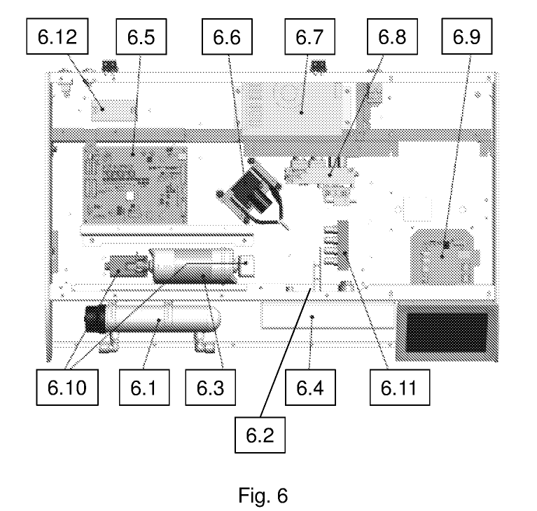

The oxygen concentration inside an incubation chamber can be regulated by adding gas such as oxygen, nitrogen, carbon dioxide, helium or another inert gas, or a mixture of two or more of these gases. A gas distribution system for incubators may comprise a gas supply, e.g. supply of N₂ and CO₂, regulated by proportional valves, and gas decontamination means such as a UV lamp, a HEPA (high efficiency particulate arresting) filter and a VOC (volatile organic compounds) filter. The gas distribution system may also comprise a gas mixing chamber, comprising an O₂ sensor and CO₂ sensor and optionally a pH monitor. When the incubator is in operation, the incubation chamber and a gas distribution system may form a closed (fluid) pipelined system, where the gas is circulated in between. A circulation means for recirculating the gas between the incubation chamber and the gas distribution system may reduce the consumption of gas during operation as new gas is only supplied to the closed recirculation system when needed to sustain the desired gas concentration. A principal diagram of one embodiment of the gas distribution system of the presently disclosed incubator is shown in FIG. 2.

To emulate the conditions in the uterus, and thereby optimize the developmental conditions for embryos stored in an incubator, a controlled atmosphere is critical. A controlled atmosphere around the embryo contained in the incubation chamber is typically obtained by the supply of gasses. The position of the gas inlet and gas outlet within the incubating chamber may affect the local gas distribution in time that will depend on diffusion. A way to ensure that the local atmosphere around the embryo is best possibly controlled, is to position the gas inlet and outlet, such that the gas flow in the incubating chamber is in-plane across the embryo. In one embodiment of the invention, the fluid communication between the gas distribution system and the incubating chamber(s) comprises a gas inlet and a gas outlet at opposite ends of the incubating chamber(s) such that the gas flow in the incubating chamber(s) is an in-plane flow across the incubating chamber(s). In a further embodiment of the invention, the gas flow to each chamber is provided by a common inlet manifold, an example of such is illustrated by the general flow diagram in FIG. 2. The conditions in a uterus may differ from woman to woman, and it may therefore be desirable to be able to control the atmosphere independently within each incubating chamber. In one embodiment of the invention, the gas flow in each incubating chamber can be controlled independently.

For management of the embryos to the incubator such as placing and removing them, access from outside is needed and may be obtained via a closure. In one embodiment of the invention, the incubating chamber comprises a chamber closure, for each incubating chamber, in order to easily access the embryos. The closure may be a lid 3.1, and an example of such is illustrated in FIG. 3. In a further embodiment, the closure is opened and closed without a locking mechanism. In a further embodiment of the invention the closure is configured for pressure equalization, such as a lid that will open shortly at a certain overpressure within the incubating chamber.

During the management of the incubator, interruptions that may affect the incubating chamber environment and the local environment around the embryo may occur. A critical point is the points in time where the incubating chamber is accessed from outside. Thus incubators with an ability of fast recovery of the environmental conditions are wanted. By incorporating additional gas flow regulation mechanisms, an incubator which recovers faster may be obtained. In one embodiment of the invention, the incubator is configured such that the gas flow within the chamber is stopped for a predefined period of time when a chamber closure is opened. This will prevent air from outside from being sucked into the incubating chamber, and the disturbance of the environment in the chamber is thereby minimized. In a further embodiment of the invention, the predefined period of gas flow stop is until the closure is closed. In a further embodiment, the predefined period of gas flow stop is until the closure is closed, or until a maximum period of e.g. 20 seconds, or between 10 and 20 seconds, has occurred.

Users of an embryo incubator may sometimes forget to close the chamber closure after gaining access to a chamber. In a further embodiment the presently disclosed incubator is therefore configured such that an audible, tangible and/or visible alarm is activated upon opening a chamber closure, and/or activated after a predefined period of time with opened chamber closure, in order to improve security of the incubator. A suitable period of time until alarm activation may be in the order of between 20 and 60 seconds, more preferably between 30 and 60 seconds, or between 1 and 2 minutes.

In one embodiment of the invention, the gas flow is increased temporarily upon closing a closure, thus acting as a flushing mechanism. The mechanism will enable faster recovery of the gas atmosphere within the incubating chamber upon interruptions. The gas flow may be increased for up to 10 seconds, such as 4 seconds, 7 seconds, or 10 seconds upon closing the closure. The increased gas flow may correspond to a system flow of maximum 10 L/min, such as 4 L/min, 6 L/min, or 8 L/min. In a preferred embodiment of the invention, the gas flow maximum is configured to be consistent with the size of the chamber.

After performing said flushing mechanism, the gas flow may be gradually reduced to a predefined level, which may be a steady state level characterized by minimized gas supply to the system for sustaining a stable atmosphere. The steady state level may correspond to a system flow of 0.5 L/min, 1 L/min, or 2 L/min. In one embodiment of the invention, the flow is decreased gradually by a stepwise reduction in flow, configured such that the predefined steady state level is obtained after maximum 30 minutes, such as 10, 20, or 30 minutes.

When placing the embryos in an incubating chamber, it is advantageous that their physical state is close to the conditions within the chamber, i.e. both their temperature and that the oxygen content is equilibrated to the atmosphere in the chamber. If the physical starting state of the embryo to be placed in the incubating chamber is significantly different from conditions within the chamber, the equilibration to the chamber conditions may cause a disturbance. However, if the embryos are prepared to the conditions within the incubator, this will minimize a possible disturbance of the conditions in the incubating chamber. In one embodiment of the invention, the incubator comprises a preparation chamber 3.2 that is an extra heated chamber for heating e.g. inserts, dishes, and media, and in fluid communication with the gas distribution system. The temperature and gas atmosphere of the preparation chamber may be controlled in the same manner as the incubation chamber. The preparation chamber may be isolated from the incubation chamber(s) by valves as illustrated in FIG. 2.

In one embodiment of the invention, the incubator comprises a user interface, which may be used to provide overview of the incubating chamber(s), display and control the environmental conditions within the chamber(s), and exchange data on the chamber(s) content, such as patient ID. The user interface may be based on a general purpose computer with a processing unit and a memory. An example of a screen shot showing an overview of the incubating chambers and the conditions within one chamber is illustrated in FIG. 4. In a further embodiment, the user interface is a touch screen 3.3, and an example of such is shown in FIG. 3. In a further embodiment of the invention, the user interface is configured to generate a temperature profile and a cyclic temperature profile. An example of a cyclic profile could be a daily variation, with a predefined repeat temperature cycle time of 24 hours. Another example of a cyclic profile could be a variation over more than one day, such as several days. In a further embodiment of the invention, the user enters multiple timer's points or time inputs, and multiple temperature set points, which are configured to define a cyclic temperature profile. In a further embodiment of the invention, the user enters four timer's points or time input, and two temperature set points, which defines the daily cycle profile, and the cycle is repeated automatically. In a further embodiment, the cycle is repeated automatically for an infinite number of times until a stop is activated, e.g. by the user. In a further embodiment, the cycle is repeated automatically for a finite and predefined number of times.

In a further embodiment of the invention, the user interface is configured to provide an output when a chamber closure is opened, and an example of an output could be the patient ID related to said chamber.

For incubators adapted to contain multiple embryos and embryos from more than one patient, it is critical to minimize risk of cross contamination and risk of mix up from patient to patient. In one embodiment of the invention, the incubator comprises an identification system. In one embodiment of the invention, the incubator comprises a wireless identification system, such as RFID. In a further embodiment, the identification system and/or the RFID system is configured to identify an embryo, an incubating chamber and patient information.

In one embodiment of the invention, the incubator comprises a top unit 5.1 and a bottom unit 5.2 interconnected by at least one hinge 5.3, the top unit comprising the incubating chamber(s), the bottom unit comprising at least parts of the gas distribution system and at least parts of the temperature control system. In a further embodiment of the invention, the hinge opening of the top unit from the bottom unit may further be supported by gas springs 5.4.

In a further embodiment of the invention, the bottom unit comprises the parts of the gas distribution system which is known to have limited lifetime and needs regular replacement, such as a UV lamp 6.1, an O₂ sensor 6.2, a HEPA filter 6.3, and/or a VOC filter. The bottom unit may optionally comprise a pH monitor, such as a touch free optic pH meter, which may be placed in the gas mixing chamber 6.4. In a further embodiment of the invention, pH monitor(s) are placed in one or more of the incubation chambers. The bottom unit may further optionally comprise components such as main board PCB 6.5, gas pump 6.6, power supply 6.7, gas control manifold 6.8, touch screen controller, e.g. android PCB unit 6.9, gas connectors 6.10, channel manifold 6.11, and UV ballast 6.12.

1. An incubator for incubating embryos, comprising:
one or more incubating chambers adapted to contain one or more embryos;
a gas distribution system in fluid communication with the one or more incubating chambers and configured to sustain a predefined level of O₂ and/or CO₂ in the one or more incubating chambers; and
a temperature control system configured to provide the one or more incubating chambers with a cyclic temperature profile.

2. The incubator according to claim 1, wherein the cyclic temperature profile simulates a daily biological temperature variation.

3. The incubator according to claim 1, wherein the cyclic temperature profile corresponds to a basal body temperature cycle of a human being.

4. (canceled)

5. (canceled)

6. The incubator according to claim 1, wherein the cyclic temperature profile is defined by a 24 hour duration, a maximum temperature, a minimum temperature, and four predefined temperature change times, and wherein the cyclic temperature profile is repeated automatically.

7. (canceled)

8. The incubator according to claim 6, wherein the cyclic temperature profile is repeated automatically for an infinite number of times until a stop is activated, or wherein the cyclic temperature profile is repeated automatically for a finite, predefined number of times.

9. (canceled)

10. The incubator according to claim 1, wherein each of the one or more incubating chambers is surrounded by a heating element that has little to no electromagnetic radiation.

11. The incubator according to claim 10, wherein each of the one or more incubating chambers is shaped as a box, wherein at least one heating element is disposed within all six sides of the box, and wherein one side of the box comprises a lid.

12. The incubator according to claim 10, wherein the heating element comprises a planar foil of insulating material on which an electrically conducting wire is positioned on or contained in a pattern that is configured to reduce or eliminate electromagnetic radiation emitted from the incubator to the surroundings.

13. The incubator according to claim 12, wherein the planar foil of insulating material is sandwiched between superjacent foil parts containing the electrically conducting wire.

14. The incubator according to claim 13, wherein directions of current in the superjacent foil parts are opposite one another across the planar foil of insulating material, such that a resultant electromagnetic field is minimized.

15. The incubator according to claim 14, wherein the electrically conducting wire follows a meandering path.

16. (canceled)

17. The incubator according to claim 14, wherein the electrically conducting wire positioned within the superjacent foil parts provides two circuit halves that are mirror images of one another such that the directions of the current in the superjacent foil parts are opposite one another in the superjacent foil parts.

18. (canceled)

19. The incubator according to claim 13, wherein the heating element comprises at least one additional layer of insulating material and an adhesive applied on at least one surface of the at least one additional layer of insulating material.

20. The incubator according to claim 1, comprising a plurality of incubating chambers that can be temperature controlled independently.

21. The incubator according to claim 20, wherein the cyclic temperature profile in each incubating chamber of the plurality of incubating chambers can be controlled independently.

22. The incubator according to claim 20, wherein the cyclic temperature profile in all of the incubating chambers of the plurality of incubating chambers can be controlled together.

23. The incubator according to claim 20, wherein the fluid communication between the gas distribution system and the plurality of incubating chambers is provided by a gas inlet and a gas outlet at opposite ends of the plurality of incubating chambers such that a gas flow in the plurality of incubating chambers is an in-plane flow across the plurality of incubating chambers.

24. The incubator according to claim 23, wherein the gas flow in each incubating chamber of the plurality of incubating chambers can be controlled independently.

25. The incubator according to claim 23, wherein the gas flow in each incubating chamber of the plurality of incubating chambers is supplied from the same manifold.

26. The incubator according to claim 1, further comprising a chamber closure providing pressure equalization for each of the one or more incubating chambers.

27. The incubator according to claim 26, wherein the incubator is configured such that the gas flow within the incubating chamber is stopped for a predefined period of time when one or more chamber closures is opened.

28. (canceled)

29. The incubator according to claim 26, wherein the incubator is configured such that an alarm is activated upon one or more chamber closures being opened and/or activated after a predefined period of time within one or more chamber closures being opened.

30. The incubator according to claim 26, wherein the gas flow is increased temporarily to a higher value upon closing the chamber closures, and then the gas flow is reduced from the higher value to a steady state value by a stepwise reduction in flow.

31. (canceled)

32. (canceled)

33. The incubator according to claim 1, further comprising a preparation chamber in fluid communication with the gas distribution system.

34. The incubator according to claim 1, further comprising an electronic user interface by which a user can generate the cyclic temperature profile.

35. (canceled)

36. The incubator according to claim 34, wherein the electronic user interface provides a visual output and/or an audible output when a chamber closure is open and/or has been opened for a predefined period of time.

37. (canceled)

38. The incubator according to claim 1, further comprising a wireless identification system.

39. The incubator according to claim 1, further comprising a top unit and a bottom unit interconnected by at least one hinge, the top unit comprising the one or more incubating chambers, and the bottom unit comprising at least parts of the gas distribution system and parts of the temperature control system.

40. (canceled)

41. The incubator according to claim 39, wherein the bottom unit comprises a UV lamp, an O₂ sensor, a HEPA filter, and/or a VOC filter.

42. The incubator according to claim 1, wherein the gas distribution system comprises a pH meter.
